(19) United States
(12) Patent Application Publication
(10) Pub. No.: US 2026/0188505 A1
(43) Pub. Date: Jul. 2, 2026

(54) PROGNOSTIC RISK ANALYSIS SYSTEM FOR HEAD AND NECK CANCER

(71) Applicant: Ever Fortune.AI CO., Ltd., Taichung City (TW)

(72) Inventor: ZHE-HAO YANG, Taichung City (TW)

(73) Assignee: Ever Fortune.AI CO., Ltd., Taichung City (TW)

(21) Appl. No.: 19/030,552

(22) Filed: Jan. 17, 2025

Related U.S. Application Data
(60) Provisional application No. 63/739,071, filed on Dec. 26, 2024.

(51) Int. Cl. G16H 50/30; G06N 20/00; G16H 10/60; G16H 30/40; G16H 50/50; G16H 50/70

(52) U.S. Cl. CPC G16H 50/30; G06N 20/00; G16H 10/60; G16H 30/40; G16H 50/50; G16H 50/70

(57) ABSTRACT

A prognostic risk analysis system for head and neck cancer includes a data collection module, a data processing module, a model training module, an analysis module, a risk stratification module and a clinical application module. The data collection module collects a training data of patients, each training data including a clinical data and an image data. The data processing module performs a preprocessing process on the training data to extract first feature values. The model training module trains a machine learning algorithm to build a prediction model by using the first feature values. The analysis module analyzes, based on the prediction model, a dataset of a patient to generate a prognostic risk data. The risk stratification module stratifies the prognostic risk data into a low-risk group, an intermediate-risk group or a high-risk group. The clinical application module applies the prognostic risk data and the risk groups to clinical practice.

### BACKGROUND OF THE INVENTION

### Technical Field

The present invention relates generally to medical analysis, and more particularly to a machine learning model based on multi medical institution big data and artificial intelligence for analyzing and predicting a clinical risk system for patients with head and neck cancer.

### Description of Related Art

Currently, the treatment of head and neck cancer primarily relies on radiation therapy, usually with a “one-size-fits-all” dosing approach. However, the approach fails to take individual differences into account, resulting in undesirable treatment effects. A number of clinical models have been developed to try to predict treatment outcomes, but most of the clinical models have not found extensive applications in clinical practice due to the lack of support from large-scale multi medical institution data. In addition, using only medical image features (such as tumor morphological features) is limited in effectively predicting treatment outcomes.

Though a number of statistical-based survival analysis models have been developed, the statistical-based survival analysis models usually focus on outcomes of a single risk (e.g., merely analyzing the overall survival of patients), and the accuracy of prediction and clinical integration are subject to limitations. For example, the existing analysis models may not be effective in comprehensively analyzing multiparameter data, such as tumor location, tumor size, clinical cancer stage, and lifestyle habits of patients with head and neck cancer. The existing analysis models also lack estimation for local tumor control and regional lymph control, and lack integration with the systems in existing medical institutions.

The present invention provides a prognostic risk analysis system for head and neck cancer to address the shortcomings of the existing techniques, significantly improving the accuracy of prognostic risk estimation for head and neck cancer and the effectiveness of clinical decision-making.

### BRIEF SUMMARY OF THE INVENTION

In view of the above, the primary objective of the present invention is to provide an analysis and prediction system for clinical risk in patients with head and neck cancer based on multi medical institution big data and an artificial intelligence machine learning model.

In addition, the present invention further involves a system, which assesses one-to-ten-year overall survival, local tumor control and regional lymph control for patients with head and neck cancer through the clinical data and the AI analysis technique for tumor medical imaging.

The present invention provides a prognostic risk analysis system for head and neck cancer configured to perform a prognostic risk analysis on a patient to be analyzed. The prognostic risk analysis system for head and neck cancer includes a data collection module, a data processing module, a model training module, an analysis module, a risk stratification module and a clinical application module. The data collection module is configured to collect a plurality of training data of a plurality of patients with head and neck cancer, each of the training data including at least one first clinical variable data and at least one first medical image data that has a tumor image. The data processing module is signally connected to the data collection module and is configured to perform a preprocessing process on the plurality of training data collected by the data collection module to extract a plurality of first feature values that respectively correspond to the plurality of first clinical variable data and the plurality of first medical image data. The model training module is signally connected to the data processing module and is configured to train a machine learning algorithm to build a prediction model by using the plurality of first feature values extracted by the data processing module. The analysis module is signally connected to the model training module and is configured to analyze, based on the prediction model, a dataset to be analyzed of the patient to be analyzed to generate a prognostic risk data of the patient to be analyzed. The risk stratification module is signally connected to the analysis module and is configured to stratify the prognostic risk data generated by the analysis module into one of a plurality of risk groups, the plurality of risk groups including a low-risk group, an intermediate-risk group and a high-risk group. The clinical application module is signally connected to the data processing module, the analysis module and the risk stratification module. The clinical application module is configured to provide a plurality of second clinical variable data and at least one second medical image data of the patient to be analyzed to the data processing module to extract a plurality of corresponding second feature values, in which the at least one second medical image data have a tumor image. The plurality of second feature values form the dataset to be analyzed of the patient to be analyzed, and the clinical application module provides the dataset to be analyzed to the analysis module to obtain the prognostic risk data of the patient to be analyzed. The risk stratification module stratifies the prognostic risk data of the patient to be analyzed into one corresponding risk group based on the prognostic risk data. Afterward, the clinical application module performs computations based on the prognostic risk output by the analysis module, the risk group output by the risk stratification module, and the plurality of second clinical variable data of the patient to be analyzed. The clinical application module outputs a clinical notification for a clinician to develop a tailored therapy plan for the patient to be analyzed based on the clinical notification.

According to the above aspect, the preprocessing process, performed by the data processing module on the plurality of training data that are collected by the data collection module, includes performing data cleaning on the plurality of training data collected by the data collection module to remove or modify missing values, error values, repeated values or extreme values of the plurality of training data; performing data standardization on the plurality of training data cleaned; and, extracting the plurality of corresponding first feature values from the plurality of training data standardized.

According to the above aspect, the prognostic risk data generated by the analysis module includes one-to-ten-year overall survival, local tumor control and regional lymph control of the patient to be analyzed. The risk stratification module stratifies each of the one-to-ten-year overall survival, the local tumor control and the regional lymph control of the patient to be analyzed into one corresponding risk group.

According to the above aspect, the one-to-ten-year overall survival, the local tumor control and the regional lymph control of the patient to be analyzed generated by the analysis module are displayed as curve graphs and tables on a screen of the clinical application module.

According to the above aspect, the clinical application module is further signally connected to a medical record database of a medical institution to automatically retrieve the plurality of second clinical variable data and the at least one second medical image data corresponding to the patient to be analyzed from the medical record database of the medical institution.

According to the above aspect, the clinical application module has an adjustment interface for the clinician capable of adjusting a high-risk threshold and a low-risk threshold, wherein the risk stratification module determines whether the prognostic risk data belongs to the high-risk group based on the high-risk threshold, and the risk stratification module determines whether the prognostic risk data belongs to the low-risk group based on the low-risk threshold. After the high-risk threshold or the low-risk threshold is adjusted, the risk stratification module re-stratifies the prognostic risk data based on the high-risk threshold adjusted or the low-risk threshold adjusted.

According to the above aspect, the clinical application module is further signally connected to the data collection module to provide the plurality of second clinical variable data and the at least one second medical image data of the patient to be analyzed to a database of the data collection module for storage. The clinical application module feeds back the prognostic risk data of the patient to be analyzed to the model training module for allowing the model training module to adjust the prediction model based on the prognostic risk data.

According to the above aspect, the present invention further includes a model validation module signally connected to the model training module. The model validation module is configured to validate the prediction model by using a validation dataset dedicated and to feed a validation result back to the model training module for allowing the model training module to adjust the prediction model based on the validation result.

According to the above aspect, the model validation module is further signally connected to the data collection module for transmitting contents of the validation dataset to a database of the data collection module for storage.

According to the above aspect, the machine learning algorithm includes one of regression analysis, decision tree, random forest, neural network or a combination thereof.

With the abovementioned design, the present invention integrates the clinical data of multiple patients with the deep learning-based model to perform the stratification and quantitative analysis of the overall survival, the local tumor control and the regional lymph control, thereby enhancing the efficiency of clinical decision-making for individual patients and the applicability of treatment methods, further supporting the development of precision medicine.

### BRIEF DESCRIPTION OF THE SEVERAL VIEWS OF THE DRAWINGS

The present invention will be best understood by referring to the following detailed description of some illustrative embodiments in conjunction with the accompanying drawings, in which

FIG. 1 is a block diagram of a prognostic risk analysis system for head and neck cancer according to a preferred embodiment of the present invention; and

FIG. 2 is a schematic view of an interface page, illustrating information displayed on a screen of a clinical application module.

### DETAILED DESCRIPTION OF THE INVENTION

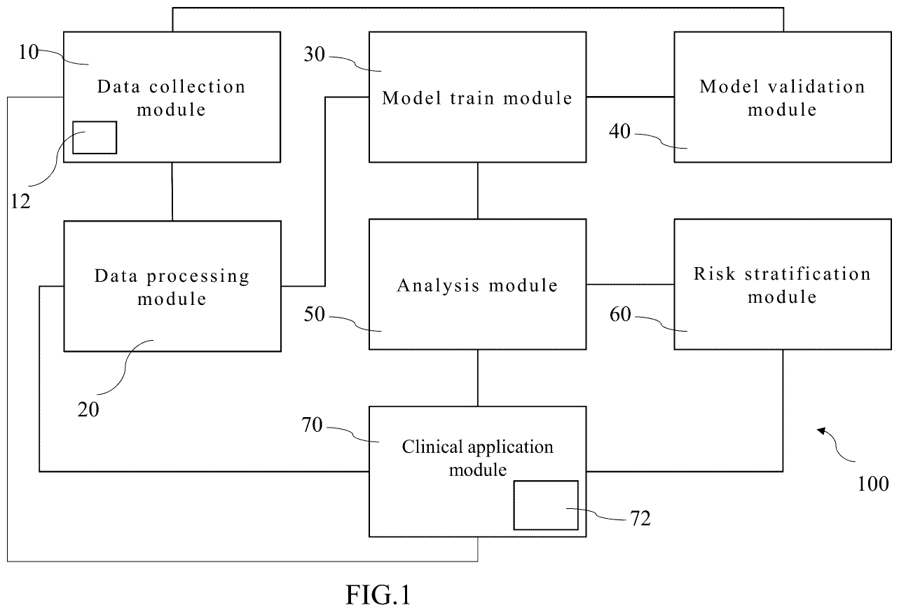

A prognostic risk analysis system 100 for head and neck cancer, configured to perform a prognostic risk analysis on a patient to be analyzed, according to a preferred embodiment of the present invention, is illustrated in FIG. 1. The prognostic risk analysis system 100 for head and neck cancer includes a data collection module 10, a data processing module 20, a model training module 30, a model validation module 40, an analysis module 50, a risk stratification module 60 and a clinical application module 70.

The data collection module 10 is configured to collect a plurality of training data of a plurality of patients with head and neck cancer, each of the training data including at least one first clinical variable data and at least one first medical image data that has a tumor image. In the current embodiment, the data collection module 10 is signally connected to medical record systems from a plurality of medical institutions, and the plurality of medical institutions could be a plurality of cancer centers and/or a plurality of medical facilities. The data collection module 10 is for collecting and integrating big data, such as the first clinical variable data and the first medical image data corresponding to medical records of the plurality of patients with head and neck cancer from the medical institutions abovementioned, into a built-in database 12 for storage. The first clinical variable data abovementioned may include, without limitation, demographic variables, medical history variables, clinical indicators, treatment variables, disease characteristics and prognostic indicators. More specifically, the demographic variables include parameters such as age, sex, or race. The medical history variables include parameters such as past history, family history, complication. The clinical indicators include parameters such as vital signs (e.g., blood pressure, heart rate, body temperature), lab results (e.g., blood analysis, imaging results). The treatment variables include parameters such as types of treatment (e.g., surgery, radiation therapy, chemotherapy), treatment dose, treatment duration. The disease characteristics include parameters such as tumor size, cancer stage, cancer grade, histological characteristics. The prognostic indicators include parameters such as overall survival, local control, and treatment response. Each of the first medical image data may include images, without limitation, ultrasound image, X-ray image, computed tomography (CT) image, magnetic resonance imaging (MRI), positron emission tomography (PET) image, and nuclear medicine image.

The data processing module 20 is signally connected to the data collection module 10. The data processing module 20 is configured to perform a preprocessing process on the plurality of training data collected by the data collection module 10 to extract a plurality of first feature values that respectively correspond to the plurality of first clinical variable data and the plurality of first medical image data. More specifically, in the current embodiment, the data processing module 20 performs data cleaning on the plurality of training data collected by the data collection module 10 to remove or modify missing values, error values, repeated values or extreme values of the plurality of training data, then performs data standardization on the plurality of training data cleaned, and extracts the plurality of corresponding first feature values from the plurality of training data standardized. The objective of the data processing module 20 is to address inconsistencies in medical records across medical institutions. If the missing values, error values, repeated values or extreme values in medical records are extracted as feature values, it can easily compromise the subsequent analysis and lead to inaccuracy. In addition, the big data from the different medical institutions is typically composed of different data fields and data values, and information of the same type may exhibit varying distributions. Therefore, using the data standardization to scale feature data to a specific range could facilitate subsequent feature extraction and analysis. In practice, the data standardization could be performed, based on the contents of information or feature requirements, by using methods such as Max-Min, MaxAbs or RobustScaler for data processing.

The model training module 30 is signally connected to the data processing module 20. The model training module 30 is configured to train a machine learning algorithm to build a prediction model by using the plurality of first feature values extracted by the data processing module 20. In practice, the machine learning algorithm used in the model training module 30 uses one of regression analysis, decision tree, random forest, neural network or a combination thereof to build the prediction model based on the contents of information or feature requirements. Given the fact that the first feature values extracted by the data processing module 20 are extracted after the data cleaning and the data standardization, the prediction model built by the model training module 30 through the feature values that are extracted by the data processing module 20 is less prone to errors and exhibits higher accuracy.

The model validation module 40 is signally connected to the model training module 30 and the data collection module 10. The model validation module 40 is configured to randomly validate the prediction model by using a validation dataset dedicated, then feed a validation result back to the model training module 30, and transmit contents of the validation dataset to the database 12 of the data collection module 10 for storage, allowing the model training module 30 to adjust the prediction model based on the validation result, further ensuring the accuracy and stability of the prediction model.

The analysis module 50 is signally connected to the model training module 30. The analysis module 50 is configured to analyze, based on the prediction model, a dataset to be analyzed of the patient to be analyzed to generate a prognostic risk data of the patient to be analyzed. More specifically, the prognostic risk data generated by the analysis module 50 includes one-to-ten-year overall survival, local tumor control and regional lymph control of the patient to be analyzed. To provide a clinician with more intuitive insights into contents of the prognostic risk data, in the current embodiment, the one-to-ten-year overall survival, the local tumor control and the regional lymph control generated by the analysis module 50 are compiled into curve graphs and probability tables for display.

The risk stratification module 60 is signally connected to the analysis module 50. The risk stratification module 60 is configured to stratify the prognostic risk data generated by the analysis module into one of a plurality of risk groups, the plurality of risk groups including a low-risk group, an intermediate-risk group and a high-risk group.

The clinical application module 70 is signally connected to the data collection module 10, the data processing module 20, the analysis module 50 and the risk stratification module 60. The clinical application module 70 is configured to provide a plurality of second clinical variable data and at least one second medical image data of the patient to be analyzed to the data processing module 20 to extract a plurality of corresponding second feature values, in which the at least one second medical image data has a tumor image. The plurality of second feature values form the dataset to be analyzed of the patient to be analyzed, and the clinical application module 70 provides the dataset to be analyzed to the analysis module 50 to obtain the prognostic risk data of the patient to be analyzed. The risk stratification module 60 stratifies the prognostic risk data of the patient to be analyzed into one corresponding risk group, i.e., into the low-risk group, the intermediate-risk group or the high-risk group, thereby achieving the objective of stratifying the patient to be analyzed into the corresponding risk group. In the current embodiment, the clinical application module 70 is signally connected to a medical record database of the medical institution, where the patient to be analyzed is currently receiving treatment, to automatically retrieve the plurality of second clinical variable data and the at least one second medical image data corresponding to the patient to be analyzed from the medical record database of the medical institution. In practice, in addition to automatic retrieval from the medical record database, the clinician could manually input relevant information to obtain the same result.

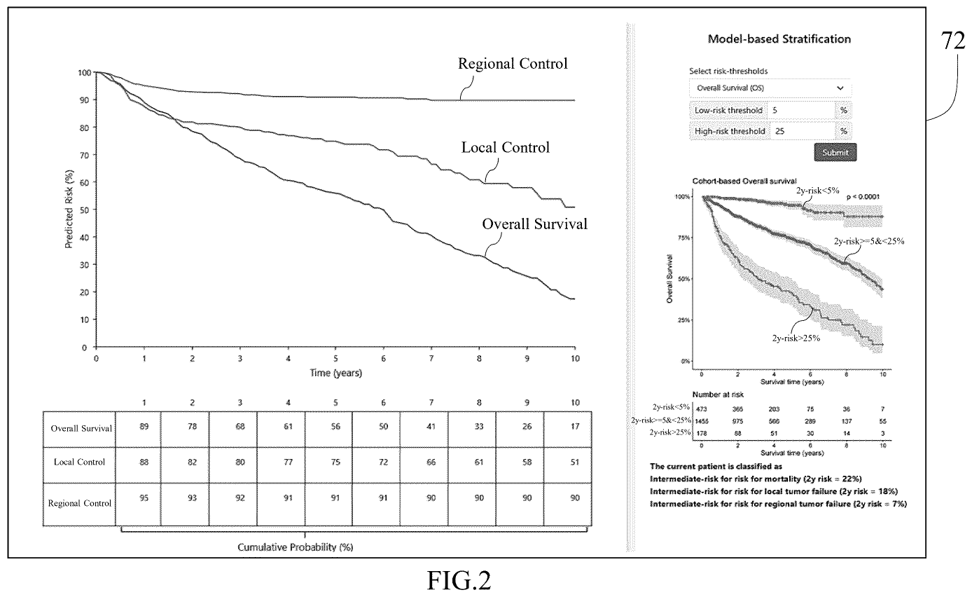

In this way, the clinical application module 70 performs computations based on the prognostic risk output by the analysis module 50, the risk group output by the risk stratification module 60, and the plurality of second clinical variable data of the patient to be analyzed, and outputs a clinical notification. The clinical application module 70 then displays the risk groups stratified, along with the curve graphs and probability tables, which are compiled from the one-to-ten-year overall survival, the local tumor control and the regional lymph control, on a screen 72 (shown in FIG. 2) of the clinical application module 70, for allowing the clinician to develop a tailored therapy plan for the patient to be analyzed based on the clinical notification.

In addition, to more effectively analyze and judge the current status of the patient to be analyzed, the clinical application module 70 has an adjustment interface displayed on the screen 72. The adjustment interface is configured for the clinician capable of adjusting a high-risk threshold and a low-risk threshold based on the current status of the patient to be analyzed, in which the risk stratification module 60 determines whether the prognostic risk data belongs to the high-risk group based on the high-risk threshold, and the risk stratification module 60 determines whether the prognostic risk data belongs to the low-risk group based on the low-risk threshold. After the high-risk threshold or the low-risk threshold is adjusted, the risk stratification module 60 re-stratifies the prognostic risk data based on the high-risk threshold adjusted or the low-risk threshold adjusted. Then, the risk stratification module 60 provides one corresponding clinical notification based on the risk group newly-stratified for the clinician to re-develop a more tailored therapy plan for the patient to be analyzed based on the clinical notification updated.

With the abovementioned design, the prognostic risk analysis system 100 for head and neck cancer of the present invention effectively and accurately assesses the one-to-ten-year overall survival, the local tumor control and the regional lymph control for the patients with head and neck cancer through the multi medical institution big data and the machine learning model together with the clinical data and the AI analysis technique for tumor medical imaging, performing the stratification and quantitative analysis of the overall survival, the local tumor control and the regional lymph control, thereby effectively assisting the clinician in improving the efficiency of clinical decision-making for individual patients and the applicability of treatment methods, further supporting the development of precision medicine.

It must be pointed out that the embodiment described above is only a preferred embodiment of the present invention. All equivalent structures which employ the concepts disclosed in this specification and the appended claims should fall within the scope of the present invention.

What is claimed is:

1. A prognostic risk analysis system for head and neck cancer, configured to perform a prognostic risk analysis on a patient to be analyzed, the prognostic risk analysis system for head and neck cancer comprising:
a data collection module, configured to collect a plurality of training data of a plurality of patients with head and neck cancer, each of the training data including at least one first clinical variable data and at least one first medical image data that has a tumor image;
a data processing module, signally connected to the data collection module, the data processing module configured to perform a preprocessing process on the plurality of training data collected by the data collection module to extract a plurality of first feature values that respectively correspond to the plurality of first clinical variable data and the plurality of first medical image data;
a model training module, signally connected to the data processing module, the model training module configured to train a machine learning algorithm to build a prediction model by using the plurality of first feature values extracted by the data processing module;
an analysis module, signally connected to the model training module, the analysis module configured to analyze, based on the prediction model, a dataset to be analyzed of the patient to be analyzed to generate a prognostic risk data of the patient to be analyzed;
a risk stratification module, signally connected to the analysis module, the risk stratification module configured to stratify the prognostic risk data generated by the analysis module into one of a plurality of risk groups, the plurality of risk groups including a low-risk group, an intermediate-risk group and a high-risk group; and
a clinical application module, signally connected to the data processing module, the analysis module and the risk stratification module, the clinical application module configured to provide a plurality of second clinical variable data and at least one second medical image data of the patient to be analyzed to the data processing module to extract a plurality of corresponding second feature values, the at least one second medical image data having a tumor image; the plurality of second feature values forming the dataset to be analyzed of the patient to be analyzed, and the clinical application module providing the dataset to be analyzed to the analysis module to obtain the prognostic risk data of the patient to be analyzed, the risk stratification module stratifying the prognostic risk data of the patient to be analyzed into one corresponding risk group based on the prognostic risk data; afterward, the clinical application module performing computations based on the prognostic risk output by the analysis module, the risk group output by the risk stratification module, and the plurality of second clinical variable data of the patient to be analyzed, and the clinical application module outputting a clinical notification for a clinician to develop a tailored therapy plan for the patient to be analyzed based on the clinical notification.

2. The prognostic risk analysis system for head and neck cancer as claimed in claim 1, wherein the preprocessing process, performed by the data processing module on the plurality of training data that are collected by the data collection module, includes:
performing data cleaning on the plurality of training data collected by the data collection module to remove or modify missing values, error values, repeated values or extreme values of the plurality of training data;
performing data standardization on the plurality of training data cleaned; and
extracting the plurality of corresponding first feature values from the plurality of training data standardized.

3. The prognostic risk analysis system for head and neck cancer as claimed in claim 1, wherein the prognostic risk data generated by the analysis module includes one-to-ten-year overall survival, local tumor control and regional lymph control of the patient to be analyzed; the risk stratification module stratifies each of the one-to-ten-year overall survival, the local tumor control and the regional lymph control of the patient to be analyzed into one corresponding risk group.

4. The prognostic risk analysis system for head and neck cancer as claimed in claim 3, wherein the one-to-ten-year overall survival, the local tumor control and the regional lymph control of the patient to be analyzed generated by the analysis module are displayed as curve graphs and tables on a screen of the clinical application module.

5. The prognostic risk analysis system for head and neck cancer as claimed in claim 1, wherein the clinical application module is further signally connected to a medical record database of a medical institution to automatically retrieve the plurality of second clinical variable data and the at least one second medical image data corresponding to the patient to be analyzed from the medical record database of the medical institution.

6. The prognostic risk analysis system for head and neck cancer as claimed in claim 1, wherein the clinical application module has an adjustment interface for the clinician capable of adjusting a high-risk threshold and a low-risk threshold, wherein the risk stratification module determines whether the prognostic risk data belongs to the high-risk group based on the high-risk threshold, and the risk stratification module determines whether the prognostic risk data belongs to the low-risk group based on the low-risk threshold; after the high-risk threshold or the low-risk threshold is adjusted, the risk stratification module re-stratifies the prognostic risk data based on the high-risk threshold adjusted or the low-risk threshold adjusted.

7. The prognostic risk analysis system for head and neck cancer as claimed in claim 1, wherein the clinical application module is further signally connected to the data collection module to provide the plurality of second clinical variable data and the at least one second medical image data of the patient to be analyzed to a database of the data collection module for storage; the clinical application module feeds back the prognostic risk data of the patient to be analyzed to the model training module for allowing the model training module to adjust the prediction model based on the prognostic risk data.

8. The prognostic risk analysis system for head and neck cancer as claimed in claim 1, further comprising a model validation module signally connected to the model training module, the model validation module configured to validate the prediction model by using a validation dataset dedicated and to feed a validation result back to the model training module for allowing the model training module to adjust the prediction model based on the validation result.

9. The prognostic risk analysis system for head and neck cancer as claimed in claim 8, wherein the model validation module is further signally connected to the data collection module for transmitting contents of the validation dataset to a database of the data collection module for storage.

10. The prognostic risk analysis system for head and neck cancer as claimed in claim 1, wherein the machine learning algorithm includes one of regression analysis, decision tree, random forest, neural network or a combination thereof
